(19) United States
(12) Patent Application Publication
(10) Pub. No.: US 2014/0146519 A1
(43) Pub. Date: May 29, 2014

(54) ILLUMINATION DEVICE HAVING AIR PURIFYING APPARATUS

(71) Applicant: Advanced Optoelectronic Technology, Inc., Hsinchu Hsien (TW)

(72) Inventors: CHUNG-MIN CHANG, Hukou (TW); PO-MIN TU, Hukou (TW); CHIEN-LIN CHANG-CHIEN, Hukou (TW); YU-WEI TSAI, Hukou (TW); HSUEN-FENG HU, Hukou (TW); CHANG-WEN SUN, Hukou (TW)

(73) Assignee: ADVANCED OPTOELECTRONIC TECHNOLOGY, INC., Hsinchu Hsien (TW)

(21) Appl. No.: 14/014,349

(22) Filed: Aug. 29, 2013

(30) Foreign Application Priority Data
Nov. 23, 2012 (CN) 201210481009X

(51) Int. Cl. A61L 9/00

(52) U.S. Cl. CPC A61L 9/00
USPC 362/96

(57) ABSTRACT

An exemplary illumination device includes a lamp and an air purifying apparatus received in the lamp. The lamp includes a base, a lamp post, a lamp cover, and a light source in the lamp cover. The air purifying apparatus is received in the base. A plurality of inlets and outlets is defined in the base. The inlets and outlets communicate with the air purifying apparatus. Air near the inlets is drawn into the air purifying apparatus via the air inlets, and the air is exhausted out of the base via the air outlets after purified by the air purifying apparatus.

### BACKGROUND

1. Technical Field

The present disclosure generally relates to lighting sources, and more particularly, to an illumination device having an air purifying apparatus therein.

2. Description of Related Art

A conventional lamp only functions as a lighting source on a desk in a house. The air in the house is easily polluted by smoke, dust, or some volatile compounds which is harmful to the health of the people working in the house. In order to purify the air in the house, an extra air purifier is needed, which may occupy more space of the house.

Therefore, what is needed is an illumination device which can overcome the limitations described above.

### BRIEF DESCRIPTION OF THE DRAWINGS

FIG. 1 is a perspective view of an illumination device in accordance with an exemplary embodiment of the present disclosure.

FIG. 2 is an exploded view of an air purifying apparatus in the illumination device of FIG. 1.

### DETAILED DESCRIPTION

An exemplary embodiment of an illumination device of the present disclosure will now be described in detail below and with reference to the drawings.

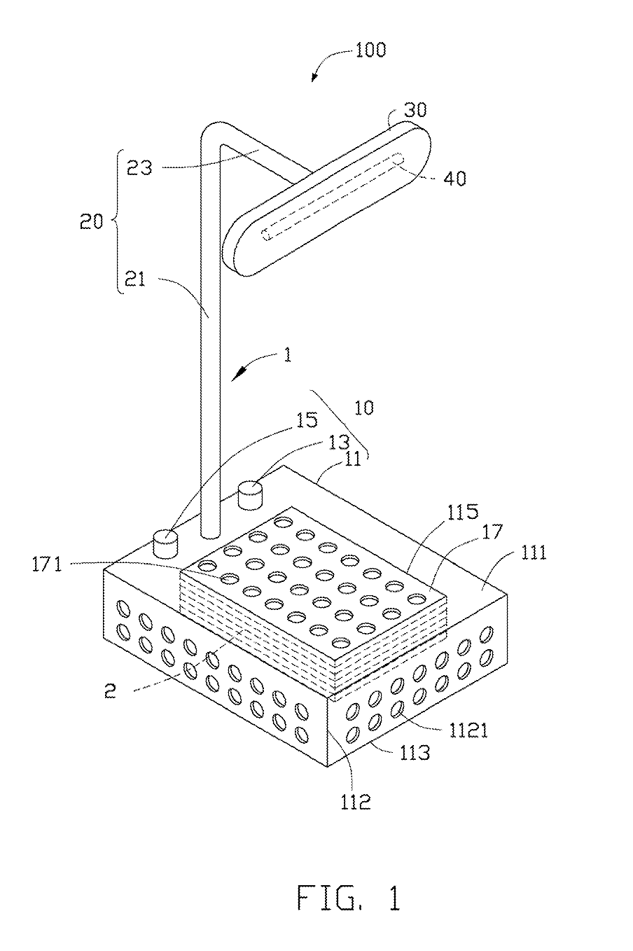

Referring to FIG. 1, an illumination device 100, in accordance with an exemplary embodiment, is provided.

The LED lamp 100 includes a lamp 1 and an air purifying apparatus 2 received in the lamp 1. A plurality of air inlets and outlets are defined in the lamp 1 to make the air purifying apparatus 2 communicate with the ambient environment. Air near the inlets is drawn into the air purifying apparatus 2 via the air inlets, and the air is exhausted out of the lamp 1 via the air outlets after purified by the air purifying apparatus 2.

The lamp 1 includes a base 10, a lamp post 20 assembled on the base 10, a lamp cover 30 fixed on an upper end of the lamp post 20, and a lighting source 40 received in the lamp cover 30.

The base 10 includes a main body 11, a first switch 13 and a second switch 15 on the main body 11.

The main body 11 is a rectangular cuboid. The main body 11 comprises a top wall 111, a bottom wall 113 opposite to the top wall 111 and a plurality of lateral walls 112 interconnecting the top wall 111 and the bottom wall 113. Alternatively, the main body 11 may be cylindrical.

An opening 115 is defined in a central portion of the top wall 111. The opening 115 is used to allow the air purifying apparatus 2 to be put in the main body 11. In this embodiment, the opening 115 is rectangular. Alternatively, the opening 115 can be circular, according to the configuration of the air purifying apparatus 2.

An upper cover 17 which substantially matches size and shape of the opening 115 is used to cover the opening 115 to seal the air purifying apparatus 2 in the main body 11. The upper cover 17 defines a plurality of first through holes 171 therein. The first through holes 171 are spaced from each other. The first through holes 171 function as the air inlets. The first through holes 171 communicate with the air purifying apparatus 2 in the main body 11. In this embodiment, the upper cover 17 is a rectangular board. Alternatively, the air purifying apparatus 2 can be received in the main body 11 without being covered by the upper cover 17, whereby the air purifying apparatus 2 is directly exposed to an outside of the base 10.

A plurality of second through holes 1121 are defined in the lateral walls 112 of the main body 11. The second through holes 1121 communicate with the air purifying apparatus 2 in the main body 11. In this embodiment, the second through holes 1121 are arranged in two rows along the lateral walls 112, and the second through holes 1121 function as the air outlets.

The first and second switches 13, 15 are located on the top wall 111 of the main body 11, and are spaced from each other. In this embodiment, the first and second switches 13, 15 are located along an edge of the top wall 111 and along a transverse direction of the main body 11.

The first switch 13 is a rotary switch, and electrically connects a power source (not shown) to turn on/off the lighting source 40 of the lamp 1. Users can turn on the lamp 1 by rotating the first switch 13 clockwise, and turn off the lamp 1 by rotating the first switch 13 counterclockwise. Alternatively, users can regulate the light intensity of the light emitted from the lighting source 40 after the lamp 1 is turned on. For example, users can increase the light intensity of the lamp 1 by rotating the first switch 13 clockwise after the lamp 1 is turned on; and users can decrease the light intensity of the lamp 1 by rotating the first switch 13 counterclockwise until the lamp 1 is turned off.

The second switch 15 is also a rotary switch, and electrically connects the power source to turn on/off the air purifying apparatus 2. Similar to the first switch 13, users can turn on air purifying apparatus 2 by rotating the second switch 15 clockwise, and turn off the air purifying apparatus 2 by rotating the second switch 15 counterclockwise. Alternatively, users can regulate the fan speed of the air purifying apparatus 2 after the air purifying apparatus 2 is turned on. For example, users can increase the fan speed of the air purifying apparatus 2 by rotating the second switch 15 clockwise after the air purifying apparatus 2 is turned on; and users can decrease the fan speed of the air purifying apparatus 2 by rotating the second switch 15 counterclockwise until the air purifying apparatus 2 is turned off.

The lamp post 20 is located on the top wall 111 of the main body 11, and extends along a direction away from the main body 11. In this embodiment, the lamp post 20 is located between the first and second switches 13, 15.

The lamp post 20 is columnar, and includes a main body 21 and a free portion 23 bent from the top end of the main body 21. The main body 21 vertically extends from the top wall 111 of the main body 11 of the base 10. A height of the main body 21 is larger than that of the main body 11 of the base 10. A length of the free portion 23 is shorter than a length of the main body 21. In this embodiment, the free portion 23 is perpendicular to the main body 21. Alternatively, the free portion 23 can be connected to the top end of the main body 21 by a universal joint, such that the free portion 23 can be pivoted around the top end of the main body 21, whereby the lighting direction of the lighting source 40 in the lamp cover 30 can be adjusted freely.

The lighting source 40 is tubular, and includes a plurality of LED chips (not shown) therein.

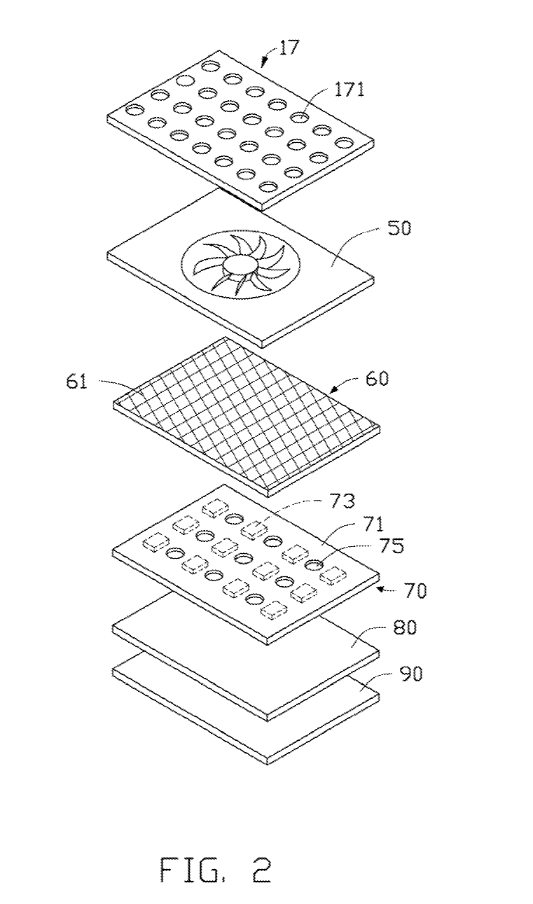

Also referring to FIG. 2, the air purifying apparatus 2 includes a fan 50, an air filter screen 60, a light emitting diode module 70, a photo catalyst 80 and an absorbent 90. The fan 50, the air filter screen 60, the light emitting diode module 70, the photo catalyst 80 and the absorbent 90 are stacked together.

The fan 50 is located under the upper cover 17, and is communicated with the first through holes 171. The fan 50 is used to draw in the air near the first through holes 171 into the air purifying apparatus 2 in the main body 11 of the base 10.

The air filter screen 60 is located under the fan 50, and defines a plurality of filtering holes 61 therein. In this embodiment, the air filter screen 60 is used to filter the solid particles with a larger size in the dust or in the smoke mixed in the air.

The LED module 70 includes a supporting plate 71, and a plurality of LED chips 73 arranged on a bottom wall of the supporting plate 71. The LED chips 73 electrically connect with a circuit (not shown) on the bottom surface of the supporting plate 71. The supporting plate 71 defines a plurality of air guiding holes 75 therein to allow the air to pass through. In this embodiment, each air guiding hole 75 is defined between two adjacent LED chips 73.

The LED chips 73 face towards the photo catalyst 80 located under the supporting plate 71. The photo catalyst 80 can be excited by the light emitted from the LED chips 73. Preferably, each LED chip 73 is an ultraviolet light emitting diode.

The photo catalyst 80 includes metal oxide nanoparticles. The photo catalyst 80 can be excited by the light from the LED chips 73 to deodorize the air, to kill germs in the air, and to decompose the harmful organic in the air. Preferably, the photo catalyst 80 includes titanium dioxide nanoparticles.

The absorbent 90 is located in the main body 11 of the base 10, and is under the photo catalyst 80. The absorbent 90 is used to absorb the harmful organic which is difficult to decompose and the particles with a smaller size in the polluted air. Preferably, the absorbent 90 includes active carbon.

When the illumination device 100 works, users control the lamp 1 and the air purifying apparatus 2 respectively and independently by manipulating the first and second switches 13, 15. When light is needed, users can control the lighting source 40 to be turned on via manipulating the first switch 13. Alternatively, users can also control the light intensity of the lighting source 40 via manipulating the first switch 13.

When the air is polluted by gases or particles harmful to health, users can turn on the air purifying apparatus 2 via manipulating the second switch 15 to purify the air. In detail, when the air purifying apparatus 2 works, the fan 50 draws the polluted air near the inlets of the base 10 efficiently into the air purifying apparatus 2. This part of air successively passes through the air filter screen 60, the air guiding holes 75 of the LED module 70, the photo catalyst 80 and the absorbent 90 to be purified.

And when the polluted air passes through the filter air screen 60, solid particles with larger size are filtered by the filter air screen 60. And then, the harmful organic in the filtered air is decomposed by the photo catalyst 80, the harmful organic which is difficult to decompose and the solid particles with smaller size mixed in the polluted air are absorbed by the absorbent 90. And, the purified air is driven by the fan 50 to exhaust out of the base 10 via the outlets in the lateral walls 112 of the main body 11.

According to the illumination device 100, firstly, because the base 10 defines a plurality of inlets in the top wall 111 and a plurality of outlets in the lateral walls 112, the polluted air can be purified more efficiently and the purified air can be provided more efficiently. Secondly, because the air purifying apparatus 2 is located in the main body 11 of the base 10, the circulation path of the air from the inlets to the outlets is shorted, such that the purifying efficiency is improved. Thirdly, because the air purifying apparatus 2 is located in the main body 11 of the base 10, the air purifying apparatus 2 does not occupy an extra space of a desk. Fourthly, the air purifying apparatus 2 in the main body 11 can be easily installed or removed, and the air purifying apparatus 2 also can be easily washed for reuse.

It is to be understood that the above-described embodiment is intended to illustrate rather than limit the disclosure. Variations may be made to the embodiment without departing from the spirit of the disclosure as claimed. The above-described embodiment illustrates the scope of the disclosure but do not restrict the scope of the disclosure.

What is claimed is:

1. An illumination device comprising:
a lamp; and
an air purifying apparatus received in the lamp, a plurality of air inlets and outlets being defined in the lamp to make the air purifying apparatus communicate with the outside of the lamp, air near the inlets being drawn into the air purifying apparatus via the air inlets, and the air being exhausted out of the lamp via the air outlets after purified by the air purifying apparatus.

2. The illumination device of claim 1, wherein the lamp comprises a base, a lamp post assembled on a top wall of the base, a lamp cover fixed on an outer end of the lamp post and a lighting source located in the lamp cover, and the air purifying apparatus is received in the base.

3. The illumination device of claim 2, wherein the base includes a top wall, a bottom wall opposite to the top wall and a plurality of lateral walls interconnecting the top wall and the bottom wall, and an opening is defined in a central portion of the top wall to allow the air purifying apparatus to be put in the base.

4. The illumination device of claim 3, further comprising an upper cover matches with the opening in the top wall, wherein the upper cover covers the opening to seal the air purifying apparatus in the base.

5. The illumination device of claim 4, wherein a plurality of first through holes are defined through the upper cover to communicate with the air purifying apparatus in the base, a plurality of second through holes are defined through the lateral walls of the base to communicate with the air purifying apparatus, the first through holes function as the air inlets, and the second through holes function as the air outlets.

6. The illumination device of claim 2, wherein the lamp post is tubular, and comprises a main body extending from the top wall of the base and a free portion bent from a top end of the main body.

7. The illumination device of claim 6, wherein the free portion can be pivoted around the top end of the main body, such that the lighting direction of the lighting source can be adjusted freely.

8. The illumination device of claim 2, wherein the lighting source comprises a plurality of LED chips.

9. The illumination device of claim 1, wherein the air purifying apparatus comprises a fan, an LED module and a photo catalyst, the photo catalyst is capable of being excited by the light emitted from the LED module to decompose harmful organic in the polluted air drawn into the air purifying apparatus by the fan via the inlets of the base.

10. The illumination device of claim 9, wherein the air purifying apparatus further comprises an absorbent, the absorbent absorbs the harmful organics which is difficult to decompose and the solid particles with smaller size mixed in the decomposed air.

11. The illumination device of claim 10, wherein the air purifying apparatus further comprises an air filter screen and the air filter screen filter the solid particles with larger size.

12. The illumination device of claim 9, wherein the LED module comprises a plurality of ultraviolet LED diodes.

13. The illumination device of claim 12, wherein the photo catalyst comprises titanium dioxide nanoparticles.

14. The illumination device of claim 10, wherein the absorbent comprises active carbon.

15. An illumination device comprising:
a lamp comprising a hollow main body, and a plurality of inlets and outlets being defined in the main body, the lamp comprising an LED light source; and
an air purifying apparatus received in the main body of the lamp and communicating with the inlets and outlets of the main body;
air being absorbed by the air purifying apparatus and drawn into air purifying apparatus from the inlets, and finally exhausted out of the main body via the outlets after the air purified by the air purifying apparatus, the air purifying apparatus comprising at least one ultraviolet light emitting diode.

16. The illumination device of claim 15, wherein the air purifying apparatus further comprises a fan, an air filter screen, a photo catalyst and an absorbent stacked together.

17. The illumination device of claim 16, wherein the photo catalyst comprises titanium dioxide nanoparticles which are used to be activated by the light from the at least one ultraviolet light emitting diode, and the absorbent comprises active carbon.
